(19) United States
(12) Patent Application Publication
(10) Pub. No.: US 2004/0180036 A1
(43) Pub. Date: Sep. 16, 2004

(54) Salts of codrugs and uses related thereto

(75) Inventors: Paul Ashton, Boston, MA (US); Tadeusz Cynkowski, Brookline, MA (US); Grazyna Cynkowska, Brookline, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA (US)

(21) Appl. No.: 10/761,494

(22) Filed: Jan. 21, 2004

Related U.S. Application Data
(60) Provisional application No. 60/441,726, filed on Jan. 21, 2003.

(51) Int. Cl. A61K 38/19; A61K 38/17; A61K 31/485

(52) U.S. Cl. 424/85.1; 514/285; 514/176; 514/12; 530/351; 530/409; 546/44; 514/263.21

(57) ABSTRACT

A salt of a codrug of at least two drug compounds covalently linked to one another via a labile bond to form a single codrug, and methods of use of the codrug salt for the treatment of various medical conditions. The codrug salt may be administered by itself or in the form of a bioerodible or nonbioerodible substance.

### CROSS-REFERENCE TO RELATED APPLICATION

The present application claims the benefit of U.S. Provisional Application No. 60/441,726, filed Jan. 21, 2003; the specification of which is hereby incorporated by reference in its entirety.

### FIELD OF THE INVENTION

The present invention is related to the field of controlled pharmaceutical delivery, especially to salt forms of codrug compounds. The present invention further relates to processes for their production, the use of these salts in the treatment of diseases, as well as to pharmaceutical preparations comprising these salts.

### BACKGROUND OF THE INVENTION

Pain is one of the most frequent signs of a disease or an injury. Though pain is to be understood as a warning and self-protective function of the organism, patients experiencing pain generally request pain-killing or at least pain-relieving substances. For this reason, one of the most important concerns in medicine is to provide pain relievers. The function of these substances, so-called analgesics, is to reduce or suppress the sensation of pain without causing a general narcotic effect. Based on their potency, therapeutic mechanism, and side effects, two groups of analgesics are generally recognized: very potent analgesics acting on the central nervous system, and low to moderately potent ones primarily having a peripheral action. Active substances acting on the central nervous system are frequently associated with a habit-forming potential that can develop into addiction. Morphine is one example of such substances. Morphine is commercially available for parenteral or peroral application to control acute posttraumatic or postoperative pain, as well as chronic pain, for example, in the state of advanced cancer.

A great variety of peripherally effective analgesics with different potency and different dosages exist today. These compounds relieve pain but do not promote healing or treat the underlying condition. Thus, these compounds typically add to the number of drugs a patient takes. Furthermore, the number of drugs a patient takes may affect the patient's compliance in consumption of pharmaceutical compositions as part of a therapeutic regimen. Patient compliance is critical for patient recovery and treatment, especially in elderly patients who may have poor memory and exhibit poor patient compliance. Other high-risk compliance groups include drug addicts, alcoholics, and those requiring long-term therapy, such as tuberculosis patients.

Thus, there is a need in the pharmaceutical arts for techniques for conveniently delivering two or more drugs in a controlled fashion.

### SUMMARY OF THE INVENTION

The present invention provides a salt of a codrug, wherein the codrug is formed by linking together two or more pharmaceutical compounds, or prodrug forms thereof, through labile covalent bonds. In general, the subject codrug salts of the present invention have a variety of advantages as a result of covalently linking the drug moieties, or prodrug forms thereof, and forming a salt. For example, the codrug salt can have different pharmacokinetic properties compared to the individual drug moieties and/or the codrug. To illustrate, the codrug salt can have a lower solubility relative to the individual drugs; the codrug salt can have a longer local residence time (e.g., as a result of decreased liver metabolism or renal clearance); the codrug salt can have a different bioavailability (e.g., due to a difference in hydrophilicity or hydrophobicity relative to individual drug components); the codrug salt can have a reduced serum protein binding activity; and a codrug salt may have improved solubility (which may, in turn, affect release rates and diffusion rates) in biological fluid as compared to the individual drug moieties and/or the codrug. Such physical characteristics as decreased solubility can be used to provide slow release of the individual drugs into the area of administration. The individual drugs are regenerated by the hydrolysis of the labile bond(s) linking the drugs together. The drugs may be linked through an ester, an amide, a carbamate, a carbonate, a cyclic ketal, a thioester, a thioamide, a thiocarbamate, a thiocarbonate, a xanthate, or a phosphate ester, etc., bond.

An additional advantage of certain embodiments of the invention is that the salt form of the codrug can be stored for a longer period than the free base form of the codrug without significant decomposition. In a preferred embodiment, the pharmaceutically acceptable salt of a codrug has a half-life at least one week longer than that of the codrug free base. In one embodiment, the codrug, as a free base, has a half-life of less than 100 hours. In another embodiment, the half-life of the codrug salt is at least one month longer than that of the codrug free base. In certain embodiments, the half-life is measured of the drug alone, while in other embodiments, the half-life may be measured of the drug in a pharmaceutical formulation, e.g., to determine the shelf life of the formulation.

Another advantage of certain embodiments of the invention is that the salt form of the codrug is less soluble in organic solvent than the codrug as a free base. The salt form of the codrug may precipitate out of organic solvents more readily than the codrug as a free base, e.g., at a purity of greater than 95% or even greater than 99%. This avoids the time and resources consuming step of separating and purifying the codrug as a free base from the individual drugs and other impurities. Additionally, the highest level of purity of the codrug as a free base obtained through chromatographic purification was 95%, less than the 99% purity that may be achieved by forming the salt of the codrug.

Additionally, the salt form of the codrug may have a different solubility in biological media than the charge-neutral form (typically greater solubility in the salt form, especially where a small, hydrophilic counterion such as a halide, mesylate, acetate, etc., is used). This change in solubility can be used to affect the rate of release of the drug, e.g., from an implant, delivery device, gel injection, etc.

One aspect of the present invention provides a salt of a codrug, wherein the codrug comprises:

a) a first drug moiety, having a first biological activity, and including a basic nitrogen in the structure of said first drug moiety,

b) a second drug moiety having a second biological activity, and

c) a linkage covalently linking said first and second drug moieties to form said codrug, said linkage being cleaved under physiological conditions to regenerate said first and second drug moieties as active agents having said first and second biological activities,

wherein the salt of the codrug has a decomposition rate at room temperature at least 50% lower than the decomposition rate of said codrug as a free base. As used herein, a basic nitrogen is a nitrogen atom for which the pKa of the conjugate acid is at least about 2, preferably at least about 5. Functional groups that typically include a basic nitrogen are amines, hydrazines, anilines, pyridines, amidines, and guandines.

In some embodiments, the first drug moiety is an opioid. In other embodiments, the first drug moiety is an opioid and the second drug moiety is a non-steroidal anti-inflammatory drug (NSAID). In some embodiments, the opioid is morphine or a morphine derivative. In certain embodiments, the second drug moiety is an antidepressant compound, an analgesic compound, a steroid, a non-steroidal antiinflammatory drug (NSAIDs), an antibiotic compound, an anti-fungal compound, an antiviral compound, an antiproliferative compound, an antiglaucoma compound, an immunomodulatory compound, a cell transport/mobility impeding agent, a cytokine, a peptide, a protein, an antimetabolite compound, an antipsoriatic compound, a keratolytic compound, an anxiolytic compound, an antipsychotic compound, an alpha-blocker compound, an anti-androgen compound, an anti-cholinergic compound, an adrenergic compound, a purinergic compound, a dopaminergic compound, a vanilloid compound, or an anti-cancer compound.

In certain embodiments, the active form of the opioid is represented in the general formula (I):

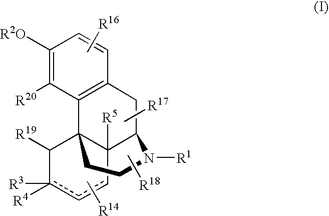

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R² represents a hydrogen, a C₁₋₆-alkyl group, or a C₁₋₆-alkanoyl group;

R³ represents a hydrogen, a C₁₋₆-alkylthio group, an aryl group, a C₁₋₆-alkoxycarbonylalkyl group, a C₁₋₆alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R¹⁴ represents a hydrogen, a lower alkyl group, a halogen group, or —C(—OH)(—R¹⁵)₂;

R¹⁵ independently for each occurrence represents a lower alkyl;

R¹⁶, R¹⁷, and R¹⁸ each independently represents a hydrogen, a lower alkyl group, or a halogen group;

R¹⁹ and R²⁰ each represents hydrogen or together represent the oxygen of a dihydrofuran ring;

Ar is phenyl or phenyl substituted by halogen, alkyl C₁₋₃, hydroxyl or alkoxy C₁₋₃; and

the dotted line indicates an optional bond.

In certain preferred embodiments, the active form of the opioid is represented in the general formula (II):

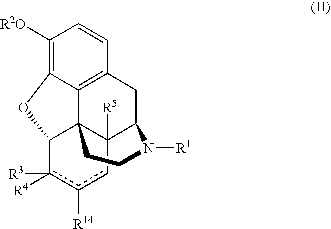

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R² represents a hydrogen, a C₁₋₆-alkyl group, or a C₁₋₆-alkanoyl group;

R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R¹⁴ represents a hydrogen, a lower alkyl group, a halogen group, or —C(—OH)(—R¹⁵)₂;

R¹⁵ independently for each occurrence represents a lower alkyl;

Ar is phenyl or phenyl substituted by halogen, alkyl C₁₋₃, hydroxyl or alkoxy C₁₋₃; and

the dotted line indicates an optional bond.

In certain embodiments, the active form of the opioid is selected from apomorphine, buprenorphine, codeine, dihydrocodeine, dihydroetorphine, diprenorphine, etorphine, hydrocodone, hydromorphone, levorphanol, meperidine, metopon, o-methylnaltrexone, morphine, naloxone, naltrexone, normorphine, oxycodone, and oxymorphone.

In some embodiments, the opioid is fentanyl or a fentanyl derivative. In other embodiments, the opioid is selected from alfentanil, β-hydroxy-3-methylfentanyl, 4-methoxymethylfentanyl, 4-methyl fentanyl, carfentanil, fentanyl, lofentanil, meperidine, remifentanil, and sufentanil.

In preferred embodiments, the active form of the opioid is an analgesic opioid.

In certain embodiments, the NSAID is selected from piroxicam, diclofenac, etodolac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib, and derivatives thereof. In some embodiments, the active form of the NSAID is diclofenac or a diclofenac derivative.

In certain embodiments, the active form of the NSAID is represented in the general formula (III):

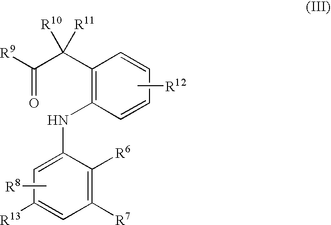

wherein

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R⁹ is OH;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo; and

R¹³ is hydrogen or trifluoromethyl when R⁶ is hydrogen or chloro and R⁷ is hydrogen or trifluoromethyl.

In certain embodiments, the codrug is represented by the general formula (IV):

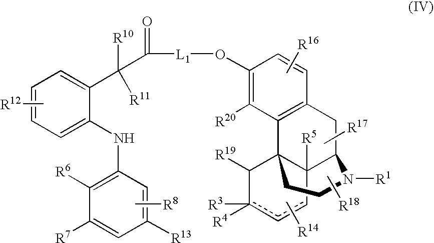

wherein

L₁ is absent or represents a linkage;

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

R¹⁵ independently for each occurrence represents a lower alkyl;

R¹⁶, R¹⁷, and R¹⁸ each independently represents a hydrogen, a lower alkyl group, or a halogen group;

R¹⁹ and R²⁰ each represents hydrogen or together represent the oxygen of a dihydrofuran ring;

Ar is phenyl or phenyl substituted by halogen, alkyl C₁₋₃, hydroxyl or alkoxy C₁₋₃; and

the dotted line indicates an optional bond.

In certain embodiments, the codrug is represented by the general formula (V):

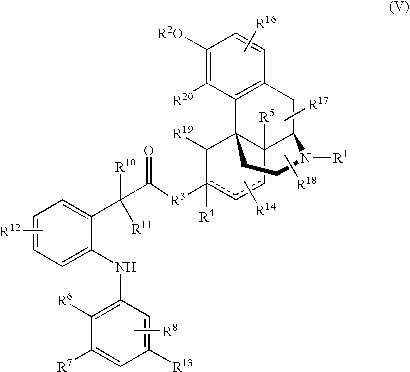

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R² represents a hydrogen, a C₁₋₆-alkyl group, or a C₁₋₆-alkanoyl group;

R³ is absent or represents a linkage;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

R¹⁵ independently for each occurrence represents a lower alkyl;

R¹⁶, R¹⁷, and R¹⁸ each independently represents a hydrogen, a lower alkyl group, or a halogen group;

R¹⁹ and R²⁰ each represents hydrogen or together represent the oxygen of a dihydrofuran ring;

Ar is phenyl or phenyl substituted by halogen, alkyl C₁₋₃, hydroxyl or alkoxy C₁₋₃; and

the dotted line indicates an optional bond.

In certain embodiments, the codrug is represented by the general formula (V):

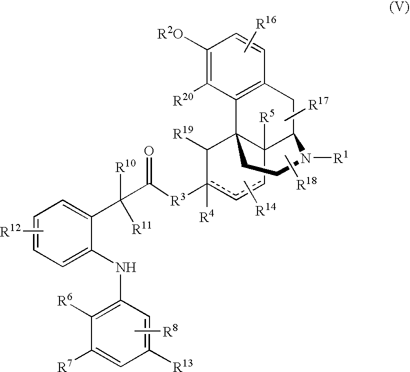

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R² represents a hydrogen, a C₁₋₆-alkyl group, or a C₁₋₆-alkanoyl group;

R³ represents a C₁₋₆-alkylthio group, an aryl group, a C₁₋₆-alkoxycarbonylalkyl group, a C₁₋₆-alkyl group, an oxygen, an azido group, a C₁₋₁₂-alkanoyl group, an amine NR^{d}₂ (wherein R^{d}, independently for each occurrence, is hydrogen, a C₁₋₆-alkyl group, or Ar), —C(═O)NH— when R⁴ is a hydrogen, or ═NO— when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

R¹⁵ independently for each occurrence represents a lower alkyl;

R¹⁶, R¹⁷, and R¹⁸ each independently represents a hydrogen, a lower alkyl group, or a halogen group;

R¹⁹ and R²⁰ each represents hydrogen or together represent the oxygen of a dihydrofuran ring;

Ar is phenyl or phenyl substituted by halogen, alkyl C₁₋₃, hydroxyl or alkoxy C₁₋₃; and

the dotted line indicates an optional bond.

In certain embodiments, the codrug is represented by the general formula (VI):

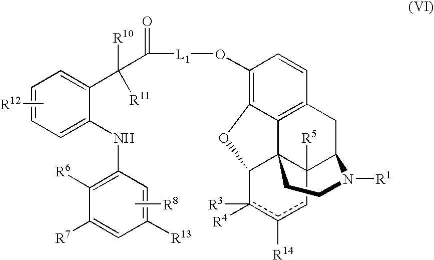

wherein

L₁ is absent or represents a linkage;

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

Ar is phenyl or phenyl substituted by halogen, alkyl C₁₋₃, hydroxyl or alkoxy C₁₋₃;

and the dotted line indicates an optional bond.

In certain embodiments, the codrug is represented by the general formula (VII):

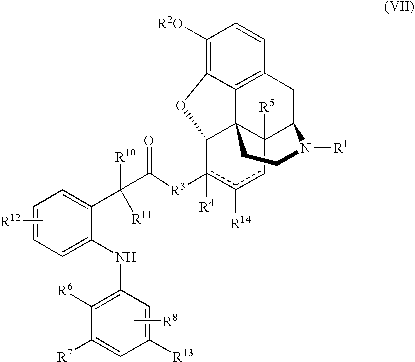

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆alkanoyl group, an Ar—C₁₋₆-alkyl group, or an allyl group;

R² represents a hydrogen, a C₁₋₆-alkyl group, or a C₁₋₆-alkanoyl group;

R³ is absent or represents a linkage;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₈, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

Ar is phenyl or phenyl substituted by halogen, alkyl C₁₋₃, hydroxyl or alkoxy C₁₋₃;

and the dotted line indicates an optional bond.

In certain embodiments, the codrug is represented by the general formula (VII):

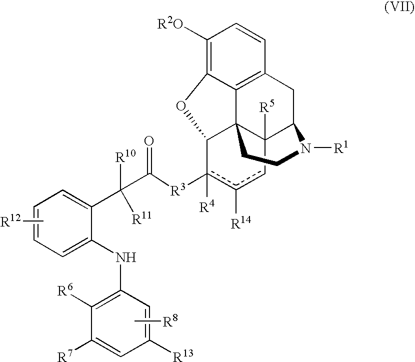

wherein

R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;

R² represents a hydrogen, a C₁₋₆-alkyl group, or a C₁₋₆-alkanoyl group;

R³ represents a C₁₋₆-alkylthio group, an aryl group, a C₁₋₆-alkoxycarbonylalkyl group, a C₁₋₆-alkyl group, an oxygen, an azido group, a C₁₋₁₂-alkanoyl group, an amine NR^{d}₂ (wherein R^{d}, independently for each occurrence, is hydrogen, a C₁₋₆-alkyl group, or Ar), —C(═O)NH— when R⁴ is a hydrogen, or ═NO— when R⁴ is absent;

R⁴ is absent or represents a hydrogen;

R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;

or R⁴ and R⁵ taken together represent —(CH₂)₂—;

R⁶ is a lower alkyl, a lower alkoxy, a fluoro, or a chloro;

R⁷ and R⁸ are each, independently for each occurrence, a hydrogen, a lower alkyl, a fluoro, a chloro, or a trifluoromethyl;

R¹⁰ is a hydrogen or a lower alkyl;

R¹¹ is a hydrogen, a lower alkyl or when R¹⁰ is hydrogen, benzyl;

R¹² is a hydrogen, a lower alkyl, a lower alkoxy, a fluoro, a chloro, or a bromo;

R¹³ is hydrogen or trifluoromethyl when R⁶ is hydrogen or chloro and R⁷ is hydrogen or trifluoromethyl;

R¹⁴ represents a hydrogen, a lower alkyl group, a halogen group, or —C(—OH)(—R¹⁵)₂;

Ar is phenyl or phenyl substituted by halogen, alkyl C₁₋₃, hydroxyl or alkoxy C₁₋₃;

and the dotted line indicates an optional bond.

In one embodiment, the first drug moiety is morphine and the second drug moiety is diclofenac.

In some embodiments, the linkage is hydrolyzed in bodily fluid. In certain embodiments, the linkage includes one or more hydrolyzable groups selected from an ester, an amide, a carbamate, a carbonate, a cyclic ketal, a thioester, a thioamide, a thiocarbamate, a thiocarbonate, a xanthate and a phosphate ester. In other embodiments, the linkage is enzymatically cleaved. In some embodiments, the linkage includes a polyethylene glycol, a glycerol, a sugar, an alkylene chain, an amino acid, or an oligopeptide.

Targeted release of the constituent drugs may also be achieved by choosing a linking bond or moiety that is selectively labile under the conditions of the target organ, e.g., an acid-labile ketal for release in the acidic environment of the stomach, a base-labile ester for release in the basic environment of the intestines, etc. In certain such embodiments, the linking bond or moiety may be selectively cleaved enzymatically by an enzyme selectively active in the target region.

In some embodiments, the codrug salt includes a counterion capable of protonating the basic amine. In certain embodiments, the codrug salt is formulated from an organic acid. The organic acid may be selected from maleic acid, malonic acid, oxalic acid, tartaric acid, citric acid, lactic acid, fumaric acid, benzoic acid, p-toluenesulfonic acid, methanesulfonic acid, acetic acid, adipic acid, formic acid, and salicylic acid. In certain embodiments, the codrug salt is formulated from an inorganic acid. The inorganic acid may be selected from hydrochloric acid, sulfuric acid, hydrobromic acid, nitric acid, and phosphoric acid.

In certain embodiments, the salt of a codrug has a decomposition rate at room temperature less than 10% of the decomposition rate of said codrug as a free base. In preferred embodiments, the salt of a codrug has a decomposition rate at room temperature less than 1% of the decomposition rate of said codrug as a free base.

In certain embodiments, the codrug has an ED₅₀ for each of said first and second biological activities at least 10 times greater than the ED₅₀ of said regenerated first and second drug moieties as active agents. In preferred embodiments, the codrug has an ED₅₀ for each of said first and second biological activities at least 1000 times greater than the ED₅₀ of said regenerated first and second drug moieties as active agents.

In certain embodiments, the codrug salt is essentially insoluble in body fluids. In preferred embodiments, the regenerated first or second drug moieties are at least 10 times more soluble than said codrug salt.

A further aspect of the present invention provides a maleic acid salt of a codrug comprising morphine covalently linked to diclofenac by a bond which is hydrolyzable to regenerate active morphine and diclofenac in vivo.

An additional aspect of the present invention provides a method for the identification of a codrug salt, comprising

i) determining the solubility of the codrug salt;

ii) determining the purity of the codrug salt; and

iii) confirming the regeneration of the first and second drug moieties as active agents.

Another aspect of the present invention provides a method of manufacturing a salt of a codrug as described herein, comprising conjugating first and second drug moieties, and crystallizing the codrug salt. Another aspect of the present invention provides a method of manufacturing a salt of a codrug as described herein, comprising crystallizing the codrug salt of a codrug.

Yet another aspect of the present invention provides a pharmaceutical composition of a codrug salt as described herein, wherein the codrug salt is combined with a pharmaceutically acceptable excipient.

An additional aspect of the present invention provides a salt of a codrug as described herein, wherein the codrug salt is dispersed in a hydrogel.

Another aspect of the present invention provides a salt of a codrug as described herein, wherein the codrug salt has a purity greater than the purity of the codrug as a free base. In certain embodiments, the codrug salt has a purity of at least 97%, more preferably at least 98%, and even more preferably at least 99%.

In some embodiments, a salt of a codrug as described above, wherein at least three drugs are linked to one another covalently.

In certain embodiments, the release of the active drugs follows pseudo-zero-order kinetics. In some embodiments, the drugs are covalently linked and release of the active drugs follows pseudo-zero-order kinetics for about 10 days to about 6 weeks. In other embodiments, the release of each active drug follows pseudo-zero-order kinetics for about 3 weeks.

In some embodiments, a salt of a codrug as described above is soluble in body fluid.

In certain embodiments, the salt form is formulated from an acid that treats at least one symptom of a condition, e.g., the deprotonated acid forms a biologically active counterion.

Another aspect of the invention provides a method of relieving pain comprising administering an effective amount of a pharmaceutically acceptable salt of a codrug as described herein to a patient in need of pain relief.

In some embodiments, the first and second drug moieties are the same. In other embodiments, the first and second drug moieties are the different.

A further aspect of the invention provides a method of targeting delivery of a codrug, wherein a salt of a codrug as described herein is converted to the codrug in a basic environment. A codrug salt may release the codrug in bodily fluids. However, the codrug may be stabilized and protonated in the acidic environment of a stomach. The codrug may pass through the stomach intact and pass into the intestines. In the intestines, the codrug may undergo deprotonation and rapid hydrolysis to release the active parent drug moieties.

### DESCRIPTION OF THE FIGURES

FIG. 1. Schematic diagram of a salt of a codrug linked by an ester bond.

FIG. 2. Schematic diagram of a salt of a codrug linked by a carbamate bond.

FIG. 3. Schematic diagram of a salt of a codrug linked by one drug attached by a cyclic ketal bond on one end of an oligoglycine linkage and another drug attached by an ester bond to the other end of the oligoglycine linkage.

FIG. 4. Schematic diagram of a salt of a codrug linked by one drug attached by a cyclic ketal bond on one end of an alkylene linkage and another drug attached by an amide bond to the other end of the alkylene linkage.

FIG. 5. Schematic diagram of a salt of a codrug linked by one drug attached by a cyclic ketal bond on one end of a poly(ethylene glycol) linkage, a second drug attached by an ester bond to a second end of the poly(ethylene glycol) linkage, and a third drug attached by a thiocarbonate bond to a third end of the poly(ethylene glycol) linkage.

### DETAILED DESCRIPTION OF THE INVENTION

The present invention is based in part on the discovery that the salt form of some codrugs is more stable than the free base form of the codrugs. The free-base form of several codrugs decomposes at a rate undesirable for a pharmaceutical compound. In one study, a free-base form of a codrug was analyzed to be 95% pure at the beginning of the study. By the end of one week, the free-base form of the codrug had decomposed to 90% purity. This rate of decomposition renders the free-base form of the codrug unsuitable for long-term storage and subsequent use. In certain embodiments, the salt form of the codrug has a shelf life at least one week longer than the free-base form of the codrug without significant decomposition, such as salts of a codrug comprising at least a morphine derivative and a non-steroidal anti-inflammatory drug.

The present method provides a solid (e.g., granular or powder) salt form of a codrug comprising at least two drug moieties selected from an antidepressant compound, an analgesic compound, a steroid, a non-steroidal antiinflammatory drug (NSAIDs), an antibiotic compound, an anti-fungal compound, an antiviral compound, an antiproliferative compound, an antiglaucoma compound, an immunomodulatory compound, a cell transport/mobility impeding agent, a cytokine, a peptide, a protein, an antimetabolite compound, an antipsoriatic compound, a keratolytic compound, an anxiolytic compound, an antipsychotic compound, an alpha-blocker compound, an anti-androgen compound, an anti-cholinergic compound, an adrenergic compound, a purinergic compound, a dopaminergic compound, a vanilloid compound, an opioids compound, and an anti-cancer compound.

Exemplary opioid compounds include morphine derivative agents, such as apomorphine, buprenorphine, codeine, desmorphine, dihydromorphine, dihydrocodeine, dihydroetorphine, diprenorphine, etorphine, ethylmorphine, heroin, hydromorphone, hydrocodone, levorphanol, meperidine, metopon, o-methylnaltrexone, morphine, naloxone, naltrexone, nalbuphine, nalorphine, normorphine, norlevorphanol, oxymorphone, oxycodone, and oxymorphone; and fentanyl or a fentanyl derivative agent, such as alfentanil, β-hydroxy-3-methylfentanyl, 4-methoxymethylfentanyl, 4-methyl fentanyl, carfentanil, fentanyl, lofentanil, meperidine, remifentanil, and sufentanil.

Suitable analgesic compounds for use as one or more constituent moieties according to the present invention include: ambucaine, benzodiazepam, benzocaine, butamben, buprenorphine, butorphanol, dezocine, dimepbeptanol, eptazocine, glafenine, isoladol, ketobenidone, p-lactophetide, lidocaine, moptazinol, metazocin, nalmefene, pentazocine, phenperidine, phenylramidol, procaine, propoxyphene, oxybuprocaine, tramadol, tetracaine, and viminol, and salts and pharmaceutically esters and prodrugs thereof.

Exemplary NSAID's include, without limitation, acetylsalicylic acid, acetaminophen, apazone, celecoxib, choline magnesium trisalicylate, diflunisal, diclofenac, etodolac, flubiprofen, fenoprofen, indomethacin, ibuprofen, ketorolac, ketoprofen, meclofenamic acid, mefenamic acid, nabumetone, naproxen, oxaprozin, piroxicam, phenylbutazone, rofecoxib, sulindac, tolmetin, and prodrugs, salts, or active metabolites thereof. Preferred NSAIDs for making codrugs are diclofenac, flurbiprofen, naproxen, and ketoprofen.

Exemplary antiproliferative agents include anthracyclines, vinca alkaloids, purine analogs, pyrimidine analogs, inhibitors of pyrimidine biosynthesis, and/or alkylating agents. Antiproliferative compounds suitable as one or more constituent moieties in the present invention include: adriamycin, alitretinoin (9-cis-retinoic acid); amifostine; arabinosyl 5-azacytosine; arabinosyl cytosine; 5-aza-2′-deoxycytidine; 6-azacytidine; 6-azauridine; azaribine; 6-azacytidine; 5-aza-2′-deoxycytidine; bexarotene (4-[1-(5,6,7,8-tetrahydro-3,5,5,8,8-pentamethyl-2-naphthalenyl)ethenyl]benzoic acid); bleomycin; capecitabine (5′-deoxy-5-fluoro-cytidine); chlorambucil; cladribine; cytarabine; cyclocytidine; daunorubicin; 3-deazauridine; 2′-deoxy-5-fluorouridine; 5′-deoxy-5-fluorouridine; docetaxel; doxorubicin; epirubicin; estramustine; etoposide; exemestane (6-methylenandrosta-1,4-diene-3,17-dione); fludarabine; fludarabin phosphate; fluorocytosine; 5-fluorouracil (5FU); 5-fluorouridine; 5-fluoro-2′-deoxyuridine (FUDR); gemcitabine; hydroxyurea; idarubicin; irinotecan; melphalan; methotrexate; 6-mercaptopurine; mitoxantrone; paclitaxel; pentostatin; N-phosphonoacetyl-L-aspartic acid; prednimustine; pyrazofurin; streptozocin; temozolomide; teniposide; 6-thioguanine; tomudex; topotecan; 5-trifluoromethyl-2′-deoxyuridine; valrubicin (N-trifluoroacetyladriamycin-14-valerate); vinorelbine; other modified nucleotides and nucleosides, and salts of the foregoing. Preferred antiproliferative agents are paclitaxel, docetaxel, methotrexate, and 5FU.

Suitable corticosteroids for use as one or more constituent moieties according to the present invention include: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difuprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, hydrocortisone, loteprednol etabonate, mazipredone, medrysone, meprednisone, methylprednisolone, methylprednisolone aceponate, mometasone furoate, paramethasone, prednicarbate, prednisolone, prednisolone 25-diethylaminoacetate, prednisolone sodium phosphate, prednisone, prednival, prednylidene, rimexolone, rofleponide, tixocortol, triamcinolone, triamcinolone acetonide, triamcinolone benetonide, and triamcinolone hexacetonide.

Illustrative examples of suitable β-lactam antibiotics include, amoxicillin, ampicillin, amylpenicillin, apalcillin, azidocillin, azlocillin, aztreonam, bacampicillin, benzylpenicillinic acid, biapenem, cefaclor, cefadroxil, cefamandole, cefatrizine, cefazedone, cefazolin, cefbuperazone, cefcapene pivoxil, cefclidin, cefdinir, cefditoren, cefepime, cefetamet, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsolodin, ceftazidime, cefteram, ceftezole, ceftibuten, ceftiofur, ceftizoxime, ceftriaxone, cefuroxime, cefuzonam, cephacetrilic acid, cephalexin, cephaloglycin, cephaloridine, cephalosporin C, cephalothin, cephamycins, cephapirinic acid, cephradine, clometocillin, cloxacillin, cyclacillin, dicloxacillin, fenbenicillin, flomoxef, floxacillin, hetacillin, imipenem, lenampicillin, loracarbef, meropenem, metampicillin, moxalactam, norcardicins (e.g., norcardicin A), oxacillin, panipenem, penicillin G, penicillin N, penicillin O, penicillin S, penicillin V, phenethicillin, piperacillin, pivampicillin, pivcefalexin, propicillin, sulbenicillin, sultamicillin, talampicillin, temocillin, ticarcillin, and tigemonam.

Antibiotic compounds suitable as one of more constituent moieties in the present invention include: metronidazole, ciprofloxacin, amikacin, tobramycin, quinolones, etc.

Antipsychotic compounds that may be used as parent compounds in the present invention include benzamides, such as amisulpride, nemonapride, and sulpiride; benzisoxazoles; butyrophenones, such as benperidol, bromperidol, droperidol, haloperidol, moperone, pipamperone, spiperone, timiperone, and trifluperidol; phothiazines, such as acetophenazine, carphenazine, dixyrazine, fluphenazine, pericyazine, perimethazine, perphenazine, piperacetazine, and pipotiazine; thioxanthenes, such as clopenthixol and flupentixol; other tricyclic antipsychotic compounds, such as carpipramine, clocapramine, mosaprimine, olanzapine, opipramol, and seroquel; and other antipsychotics, such as buramate, penfluridol, pimozide, and ziprasidone.

Anxiolytic compounds that may be used as parent compounds in the present invention include arylpiperazines, such as enciprazine and flesinoxan; benzodiazepine derivatives, such as chlordiazepoxide, clorazepate, flutazolam, lorazepam, mexazolam, nordazepam, and oxazepam; carbamates, such as emylcamate, hydroxyphenamate, meprobamate, phenprobamate, and tybamate; other anxiolytic compounds, such as benzoctamine, glutamic acid, hydroxyzine, mecloralurea, mephenoxalone, and oxanamide; and selective serotonin uptake inhibitors (SSRI's), such as fluoxetine, fluvoxamine, indalpine, indeloxazine HCl, milnacipran, paroxetine, and sertraline.

Antimetabolite compounds interfere with the normal metabolic processes within cells, e.g., by combining with the enzymes responsible for them. Antimetabolite compounds suitable as one or more constituent compounds in the present invention include: 5-fluorouracil, methotrexate, 5-fluoro-2′-deoxyuridine (FUDR), Ara-C (cytarabine), gemcitabine, mercaptopurine, and other modified nucleotides and nucleosides. Antipsoriatic compounds suitable as one or more constituent moieties in the present invention include: retinoids (including but not limited to retinoic acid, acitretin and tazarotene), salicylic acid (monoammonium salt), anthralin, 6-azauridine, vitamin D derivatives (including but not limited to calcipotriene and calcitriol), pyrogallol, and tacalcitol.

Antiandrogen compounds suitable as one of more constituent moieties in the present invention include luteinizing hormone-releasing hormone (LHRH) agonists or progestational agents, bicalutamide, bifluranol, cyproterone, flutamide, nilutamide, osaterone, oxendolone, etc., and salts and pharmaceutically esters and prodrugs thereof.

Alpha-blocker compounds suitable as one of more constituent moieties in the present invention include naftopidol and analogs of phenoxybenzamine and prazosin, and salts and prodrugs thereof.

Anti-cholinergic compounds suitable as one of more constituent moieties in the present invention include biperiden, procyclidin, trihexylphenidyl hydrochloride, atropine, ipratropium bromide, oxitropium bromide, etc., and salts and prodrugs thereof.

Adrenergic compounds suitable as one of more constituent moieties in the present invention include acebutolol, atenolol, betaxolol, timolol, propanolol, etc., and salts and prodrugs thereof.

The salt form of a codrug may be formed utilizing an acid that treats at least one symptom of a condition, for example, ascorbic acid. The salt form of a codrug may also be formed utilizing an organic acid including, without limitation, maleic acid, malonic acid, oxalic acid, tartaric acid, citric acid, lactic acid, fumaric acid, benzoic acid, p-toluenesulfonic acid, methanesulfonic acid, acetic acid, adipic acid, formic acid, and salicylic acid. Inorganic acids may also be utilized including, without limitation, hydrochloric acid, sulfuric acid, hydrobromic acid, nitric acid, and phosphoric acid. Preferably the acid will have an acid constant (pKₐ)≦4.5 in water.

The present invention provides a means of improving the pharmaceutical and pharmacological properties of pharmacologically active compounds or prodrugs by linking them together to form a codrug.

Codrug salts may be formed by conjugating two or more therapeutic agents, or prodrug forms thereof, via a labile linkage and combining a codrug conjugate with an acid, such as an acid that also possesses therapeutic qualities. A codrug conjugate may be formed by linking a first point of attachment on a first drug portion to the second point of attachment on the second drug portion through a reversible covalent linkage. A point of attachment may be a substituted or unsubstituted carbon or heteroatom including, without limitation, O, N, C, and S. The linkage may include, without limitation, a polyethylene glycol, a glycerol, a sugar, an alkylene chain, an amino acid, or an oligopeptide.

Codrug conjugates may be linked via reversible covalent bonds such as ester, amide, carbamate, carbonate, cyclic ketal, thioester, thioamide, thiocarbamate, thiocarbonate, xanthate and phosphate ester bonds, so that at the required site in the body they are cleaved to regenerate the active forms of the drug compounds. Bonds may be, but are not limited to, the type

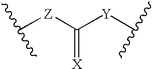

wherein Z is O, N, CH₂, CH₂O, or CH₂S, Y is O or N, and X is O or S. The rate of cleavage of the two drugs can be controlled by the type of bond, the choice of drugs, and the physical form of the conjugate. The bond may be selected from enzymatically or chemically labile bonds. The bond selected may be enzyme-specific. The codrugs are labile in water, serum, or other bodily fluids, and preferably regenerate the active parent drugs. The present invention provides a salt form of a combination of two or more drugs in a codrug with improved pharmaceutical properties that generates two active drug compounds and exhibits improved storage properties.

In an embodiment of the present invention, codrug salts provide controlled or sustained release for a systemic or local pharmacologic or physiologic effect relating to chronic pain; arthritis; and/or rheumatic conditions. A wide variety of disease states may be treated using the codrug salts of the present invention. Such disease states are known to those of ordinary skill in the art (see Goodman and Gilman, The Pharmacological Basis of Therapeutics, 8th Ed., Pergamon Press, NY, 1990; and The Merck Index, 11th Ed., Merck and Co., Inc., Rahway, N.J. 1989; incorporated herein by reference in their entireties).

Codrug salt formulations may comprise a number of other constituents to optimize release, bioavailability, or appearance and may be used in sustained release devices or systems. Such constituents are known to those of ordinary skill in the art and for example are set forth in Remington's Pharmaceutical Sciences, 18th Ed., Mack Publishing Co., Easton, Pa., 1990.

Another embodiment of the present invention comprises a codrug salt in a nonerodible matrix or reservoir system containing natural or synthetic polymers that are biologically compatible with and essentially insoluble in body fluids. Such materials include for example, but are not limited to polyvinyl acetate, polyvinyl alcohol, cross-linked polyvinyl butyrate, ethylene ethyl acrylate copolymer, polyethyl hexyl acrylate, polyvinyl chloride, polyvinyl acetals, plasticized ethylene vinyl acetate copolymer, ethylene vinyl chloride copolymer, polyvinyl esters, polyvinyl butyrate, polyvinyl formal, polyamides, polymethyl-methacrylate, polybutyl methacrylate, plasticized polyvinyl chloride, plasticized nylon, plasticized soft nylon, plasticized polyethylene terephthalate, natural rubber, polyisoprene, polyisobutylene, polybutadiene, polyethylene, polytetrafluoroethylene, polyvinylidine chloride, polyacrylonitrile, cross-linked polyvinyl pyrrolidone, polytrifluorochloroethylene, chlorinated polyethylene, poly(1,4,-isopropylidne diphenylene carbonate), vinylidine chloride, acrylonitrile copolymer, vinyl chloride-diethyl fumarate copolymer, silicone rubbers (especially medical grade polydimethylsiloxanes), ethylene-propylene rubber, silicone-carbonate copolymers, vinylidine chloride-vinyl chloride copolymer, vinyl chloride-acrylonitrile copolymer, and vinylidine chloride acrylonitrile copolymer.

In another embodiment, a totally bioerodible sustained release system for pharmacologically active agents is composed of codrug salt alone (either solid, liquid, or colloidal). Injectable codrug salt systems have a variety of applications including, but not limited to arthritis.

A codrug salt of the invention may be administered in injectable form selected from liposomes, liquids, suspensions, microspheres, and nanoparticles. Preparation of such aqueous solutions, liposomes, emulsions, and suspensions are known to those of ordinary skill in the art (see Remington's Pharmaceutical Sciences, 18th Ed., Mack Publishing Co., Easton, Pa., 1990, pp. 1504-1712, incorporated herein by reference).

Another embodiment of the invention provides a totally bioerodible sustained release system for a salt of a codrug in a formulation with another bioerodible substance such as polyvinyl acid, polyanhydride, collagen, or poly(alkylcyanoacrylate)s such as poly(butylcyanoacrylate).

Examples of a salt of a codrug of the present invention include the maleic acid salt of morphine covalently linked to diclofenac and the malonic acid salt of hydromorphone covalently linked to diclofenac. These codrug salts are stable in solid form, but labile when dissolved in bodily fluids and are rapidly hydrolyzed to regenerate the two active parent drugs.

Pellets of codrug salts of the invention may, therefore, slowly release drugs in solution or bodily fluids due to the low solubility of the conjugated forms. Pellets may be formulated from the codrug salt compounds alone or with implantable, bioerodible substances selected from polylactic acid and polyglycolic compounds. Pellets may be formulated by methods known in the art and may contain 0.1 to about 100% of the codrug salt.

Codrug salts may also be formulated in bioerodible or nonbioerodible delivery systems to further control their release. Such bioerodible systems include polylactic acid (bioerodible) to form a film around, or a matrix with a codrug salt to further improve the pharmaceutical properties. Codrug salts can be formulated in solutions of 2, 5 and 10% polylactic acid.

Amongst the advantages of codrug salt systems are that frequently no polymers are required to control release so that the devices can be extremely small (e.g., small enough to be fitted onto a haptic of an intraocular lens). Codrugs salt systems can also be formulated as suspensions (e.g., nanoparticle size range), and upper size limitations are only imposed by the intended application method. In such polymer-free compositions, there are also no concerns of residual polymer after drug has been released, nor of polymer-related toxicity.

Another aspect of the invention provides a salt of a codrug formed by linking together two or more pharmaceutical compounds, or prodrug forms thereof, through labile covalent bonds, wherein the counterion may be provided by an erodible or non-erodible polymer. Suitable polymers may be acidic and biologically compatible.

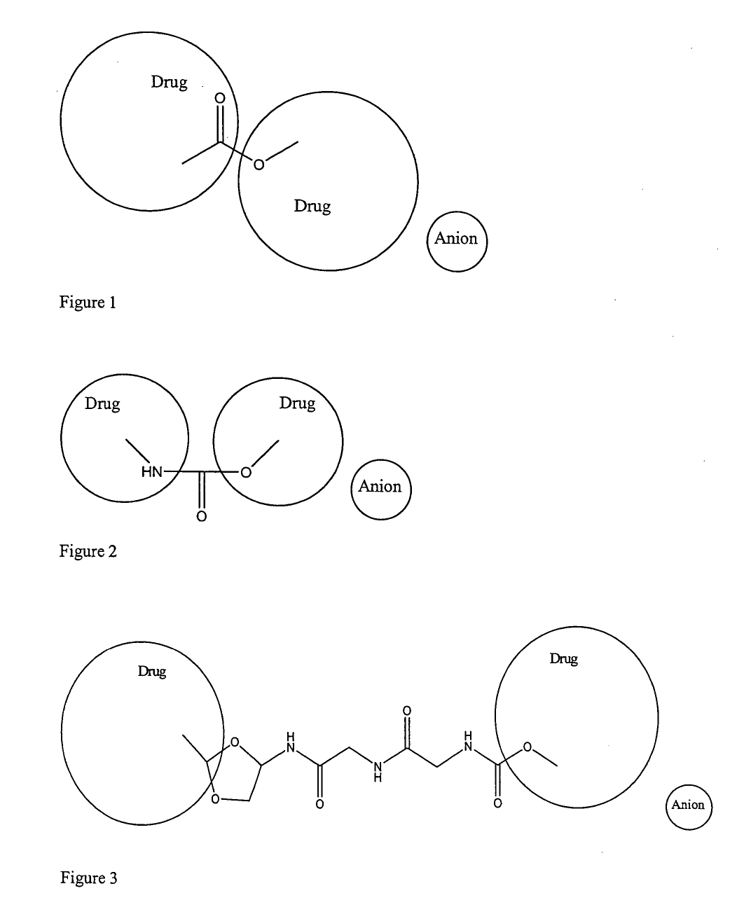

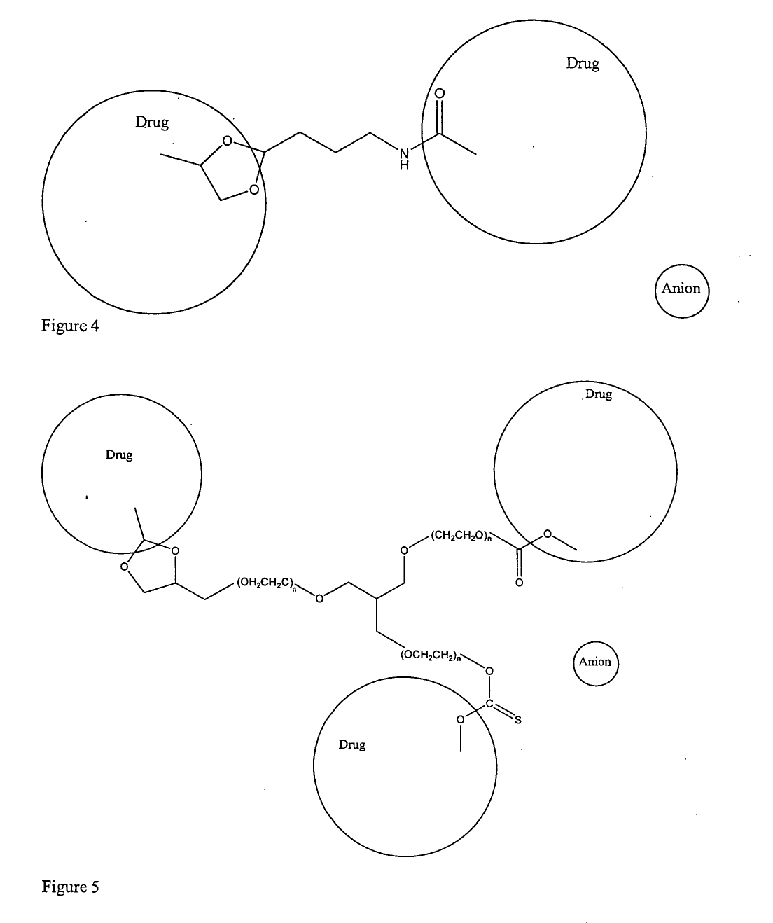

The following patents are incorporated by reference: U.S. Pat. No. 5,219,851 (col. 1, ll. 10-37), U.S. Pat. No. 3,969,519 (col. 7, ll. 21-35), U.S. Pat. No. 4,241,067 (col. 1, ll. 10-56), U.S. Pat. No. 5,317,022 (FIG. 1 and col. 1, ll. 1-22), U.S. Pat. No. 5,457,110 (col. 14, ll. 1-20), and U.S. Pat. No. 3,558,690 (col. 1, line 47-col. 2, line 20).

Definitions

The term “point of attachment” as used herein refers to a carbon atom or a heteroatom that is part of the first or second drug moiety that is able to undergo a reaction to link the two drug moieties together.

The terms “half-life” or “half-lives” refer to the time required for half of a quantity of a substance to be converted into another chemically distinct species in vitro or in vivo.

The term “fixed” as used herein means secured or attached.

The term “active” as used herein means therapeutically or pharmacologically active.

The term “ED₅₀” means the dose of a drug that produces 50% of its maximum response or effect.

The term “IC₅₀” means the dose of a drug that inhibits a biological activity by 50%.

The term “LD₅₀” means the dose of a drug that is lethal in 50% of test subjects.

The term “therapeutic index” refers to the therapeutic index of a drug defined as LD₅₀/ED₅₀.

A “patient” or “subject” to be treated by the subject method can mean either a human or non-human animal.

“Physiological conditions” describe the conditions inside an organism, i.e., in vivo. Physiological conditions include the acidic and basic environments of body cavities and organs, enzymatic cleavage, metabolism, and other biological processes, and preferably refer to physiological conditions in a vertebrate, such as a mammal.

By “opioid” it is meant any exogenous substance which binds specifically to any of several subspecies of opioid receptors. This term is used to designate a group of drugs that are, to varying degrees, opium or morphine-like in their properties, and includes morphine, analgesic morphine derivatives, and synthetic drugs producing a morphine-like effect. The pharmacological properties and therapeutic uses of the analgesics included within the classification of opioids are described in detail in Goodman and Gilman, “Opioid Analgesics and Antagonists”, The Pharmacological Basis of Therapeutics, 6th Ed., Ch. 22 (1980), incorporated by reference herein.

The term “morphine derivative” refers to morphine and all biologically active structures having a structure similar to morphine, for example, a substituted structure having at least four interconnected rings, a benzene ring, a piperidine ring, a cyclohexane ring, and a cyclohexene or cyclohexane ring. The structure may be substituted by one or more of the substitutents listed below. The morphine derivative includes without limitation morphine, codeine, hydromorphone, hydrocodone, oxymorphone, oxycodone, levorphanol, methadone, meperidine, heroin, dihydrocodeine, metopon, apomorphine, normorphine, etorphine and buprenorphine, and derivatives thereof.

A “substitution” or “substituent” on a small organic molecule generally refers to a valency on a multivalent atom occupied by a moiety other than hydrogen, e.g., a position on a chain or ring exclusive of the member atoms of the chain or ring. Such moieties include those defined herein and others as known in the art, for example, halogen, alkyl, alkenyl, alkynyl, azide, haloalkyl, hydroxyl, carbonyl (such as carboxyl, alkoxycarbonyl, formyl, ketone, or acyl), thiocarbonyl (such as thioester, thioacetate, or thioformate), alkoxyl, phosphoryl, phosphonate, phosphinate, amine, amide, amidine, imine, cyano, nitro, azido, sulfhydryl, alkylthio, sulfate, sulfonate, sulfamoyl, sulfonamido, sulfonyl, silyl, ether, cycloalkyl, heterocyclyl, heteroalkyl, heteroalkenyl, and heteroalkynyl, heteroaralkyl, aralkyl, aryl or heteroaryl. It will be understood by those skilled in the art that certain substituents, such as aryl, heteroaryl, polycyclyl, alkoxy, alkylamino, alkyl, cycloalkyl, heterocyclyl, alkenyl, alkynyl, heteroalkyl, heteroalkenyl, and heteroalkynyl, can themselves be substituted, if appropriate. This invention is not intended to be limited in any manner by the permissible substituents of organic compounds. It will be understood that ‘substitution’ or ‘substituted with’ includes the implicit proviso that such substitution is in accordance with permitted valence of the substituted atom and the substituent, and that the substitution results in a stable compound, e.g., which does not spontaneously undergo transformation such as by rearrangement, cyclization, elimination, hydrolysis, etc.

The terms ‘amine’ and ‘amino’ are art-recognized and refer to both unsubstituted and substituted amines as well as ammonium salts, e.g., as can be represented by the general formula:

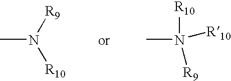

wherein R₉, R₁₀, and R′₁₀ each independently represent hydrogen or a hydrocarbon substituent, or R₉ and R₁₀ taken together with the N atom to which they are attached complete a heterocycle having from 4 to 8 atoms in the ring structure. In preferred embodiments, none of R₉, R₁₀, and R′₁₀ is acyl, e.g., R₉, R₁₀, and R′₁₀ are selected from hydrogen, alkyl, heteroalkyl, aryl, heteroaryl, carbocyclic aliphatic, and heterocyclic aliphatic. The term ‘alkylamine’ as used herein means an amine group, as defined above, having at least one substituted or unsubstituted alkyl attached thereto. Amino groups that are positively charged (e.g., R′₁₀ is present) are referred to as ‘ammonium’ groups. In amino groups other than ammonium groups, the amine is preferably basic, e.g., its conjugate acid has a pKₐ above 7.

The terms ‘amido’ and ‘amide’ are art-recognized as an amino-substituted carbonyl, such as a moiety that can be represented by the general formula:

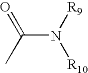

wherein R₉ and R₁₀ are as defined above. In certain embodiments, the amide will include imides. In general, when the oxygen of the above formula is replace by sulfur, the formula represents a ‘thioamide’.

The term ‘carbonyl’ is art-recognized and includes such moieties as can be represented by the general formula:

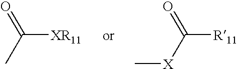

wherein X is a bond or represents an oxygen or a sulfur, and R₁₁ represents a hydrogen, hydrocarbon substituent, or a pharmaceutically acceptable salt, R_{1-11′} represents a hydrogen or hydrocarbon substituent. Where X is an oxygen and R₁₁ or R_{11′} is not hydrogen, the formula represents an ‘ester’. Where X is an oxygen, and R₁₁ is as defined above, the moiety is referred to herein as a carboxyl group, and particularly when R₁₁ is a hydrogen, the formula represents a ‘carboxylic acid’. Where X is an oxygen, and R₁₁ is hydrogen, the formula represents a ‘formate’. In general, where the oxygen atom of the above formula is replaced by sulfur, the formula represents a ‘thiocarbonyl’ group. Where X is a sulfur and R₁₁ or R_{11′} is not hydrogen, the formula represents a ‘thioester.’ Where X is a sulfur and R₁₁ is hydrogen, the formula represents a ‘thiocarboxylic acid.’ Where X is a sulfur and R_{11′} is hydrogen, the formula represents a ‘thioformate.’ On the other hand, where X is a bond, R₁₁ is not hydrogen, and the carbonyl is bound to a hydrocarbon, the above formula represents a ‘ketone’ group. Where X is a bond, R₁₁ is hydrogen, and the carbonyl is bound to a hydrocarbon, the above formula represents an ‘aldehyde’ or ‘formyl’ group.

‘Carbamate’ refers to the group having the following general structure

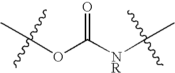

wherein R represents hydrogen or a hydrocarbon substituent.

A ‘thiocarbamate’ refers to a variant of the above group wherein the oxygen of the carbonyl is replaced by sulfur.

‘Carbonate’ refers to the group having the following general structure of

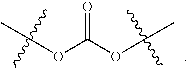

A ‘thiocarbonate’ refers to a variant of the above structure wherein the oxygen of the carbonyl is replaced by sulfur.

‘Cyclic ketal’ refers to a cyclic aliphatic group including two oxygen atoms, such as moieties having one of the following general structures:

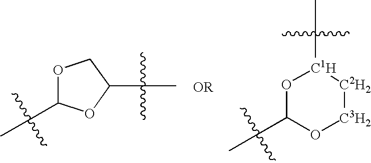

wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Phosphate ester’ has refers to a group having the following general structure

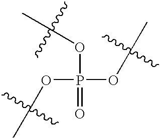

wherein each of the groups attached to the oxygens may be hydrogen, hydrocarbon, or a counterion (such as sodium) or other substituents as defined above.

A cyclic phosphate ester has the following general structure

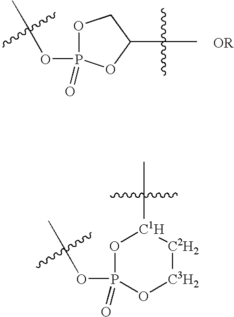

wherein substituents, such as the one depicted on C¹, could also, alternatively or additionally, be present at any other position(s) on the ring, such as on C² or C³, and/or two substituents can be present on the same position of the ring. Two carbons of the three carbons, C¹, C², and C³, together may be included in another ring structure having from 4 to 8 atoms in the ring structure.

‘Guanidino’ refers to a group having the following general structure

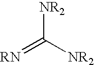

wherein each R may be, independently for each occurrence, a hydrogen or a hydrocarbon substituent. Two R's taken together may form a ring. The general structure may thus be part of one ring or a polycyclic structure.

‘Amidines’ are represented by the general formula

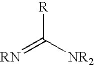

and are basic groups wherein each R may be, independently for each occurrence, a hydrogen or a hydrocarbon substituent. Two R taken together may form a ring.

‘Hydrocarbon substituents’ are moieties that include at least one C—H bond, and include groups such as alkyl, heteroalkyl, aryl, heteroaryl, carbocyclic aliphatic, and heterocyclic aliphatic groups.

‘Heteroatom’ refers to a multivalent non-carbon atom, such as a boron, phosphorous, silicon, nitrogen, sulfur, or oxygen atom, preferably a nitrogen, sulfur, or oxygen atom. Groups containing more than one heteroatom may contain different heteroatoms.

‘Heterocyclic aliphatic ring’ is a non-aromatic saturated or unsaturated ring containing carbon and from 1 to about 4 heteroatoms in the ring, wherein no two heteroatoms are adjacent in the ring and preferably no carbon in the ring attached to a heteroatom also has a hydroxyl, amino, or thiol group attached to it. Heterocyclic aliphatic rings are monocyclic, or are fused or bridged bicyclic ring systems. Monocyclic heterocyclic aliphatic rings contain from about 4 to about 10 member atoms (carbon and heteroatoms), preferably from 4 to 7, and most preferably from 5 to 6 member atoms in the ring. Bicyclic heterocyclic aliphatic rings contain from 8 to 12 member atoms, preferably 9 or 10 member atoms in the ring. Heterocyclic aliphatic rings may be unsubstituted or substituted with from 1 to about 4 substituents on the ring. Preferred heterocyclic aliphatic ring substituents include halo, cyano, lower alkyl, heteroalkyl, haloalkyl, phenyl, phenoxy or any combination thereof. More preferred substituents include halo and haloalkyl. Heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, hydantoin, oxazoline, imidazolinetrione, triazolinone, quinoline, phthalazine, naphthyridine, quinoxaline, quinazoline, quinoline, pteridine, carbazole, carboline, phenanthridine, acridine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. Preferred heterocyclic aliphatic rings include piperazyl, morpholinyl, tetrahydrofuranyl, tetrahydropyranyl and piperidyl. Heterocycles can also be polycycles.

‘Heteroalkyl’ is a saturated or unsaturated chain of carbon atoms and at least one heteroatom, wherein no two heteroatoms are adjacent. Heteroalkyl chains contain from 1 to 18 member atoms (carbon and heteroatoms) in the chain, preferably 1 to 12, more preferably 1 to 6, more preferably still 1 to 4. Heteroalkyl chains may be straight or branched. Preferred branched heteroalkyl have one or two branches, preferably one branch. Preferred heteroalkyl are saturated. Unsaturated heteroalkyl have one or more double bonds and/or one or more triple bonds. Preferred unsaturated heteroalkyl have one or two double bonds or one triple bond, more preferably one double bond. Heteroalkyl chains may be unsubstituted or substituted with from 1 to about 4 substituents unless otherwise specified. Preferred heteroalkyl are unsubstituted. Preferred heteroalkyl substituents include halo, aryl (e.g., phenyl, tolyl, alkoxyphenyl, alkoxycarbonylphenyl, halophenyl), heterocyclyl, heteroaryl. For example, alkyl chains substituted with the following substituents are heteroalkyl: alkoxy (e.g., methoxy, ethoxy, propoxy, butoxy, pentoxy), aryloxy (e.g., phenoxy, chlorophenoxy, tolyloxy, methoxyphenoxy, benzyloxy, alkoxycarbonylphenoxy, acyloxyphenoxy), acyloxy (e.g., propionyloxy, benzoyloxy, acetoxy), carbamoyloxy, carboxy, mercapto, alkylthio, acylthio, arylthio (e.g., phenylthio, chlorophenylthio, alkylphenylthio, alkoxyphenylthio, benzylthio, alkoxycarbonylphenylthio), amino (e.g., amino, mono- and di-C1-C3 alkylamino, methylphenylamino, methylbenzylamino, C1-C3 alkylamido, carbamamido, ureido, guanidino).

“Pharmaceutically acceptable salt” refers to a cationic salt formed at any acidic (e.g., hydroxamic or carboxylic acid) group, or an anionic salt formed at any basic (e.g., amino or guanidino) group. Such salts are well known in the art. See e.g., PCT Publication 87/05297, incorporated herein by reference. Such salts are made by methods known to one of ordinary skill in the art. It is recognized that the skilled artisan may prefer one salt over another for improved solubility, stability, formulation ease, price and the like. Determination and optimization of such salts is within the purview of the skilled artisan's practice. Preferred anions include halides (such as chloride), sulfonates, carboxylates, phosphates, therapeutically active carboxylates, and the like.

A “xanthate” refers to the group having the following general structure

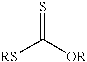

wherein R represents a hydrocarbon substituent.

Process for Making Codrug Salt

In general, a subject method includes forming an acid salt precipitate of a codrug from a solution comprising the codrug and an acid. The acid precipitate of the codrug along with a pharmaceutically acceptable carrier can then be formed into a solid tablet.

A. Precipitation of the Codrug Salts

The codrug salt precipitate can be formed as follows. The absolute concentrations in this embodiment are merely exemplary, and can be varied as determined by routine experimentation. A drug portion as a free base may be dissolved in an organic solvent. If the starting material is a salt, such as a hydrochloride salt, the free base can be formed by any suitable technique as is well known in the art, such as extraction of a drug salt suspension in MTBE (e.g., about 0.25 M) with aqueous NaOH, followed by concentration of the resultant free base by vacuum distillation. To the free base solution in the solvent is added a second drug portion under suitable reaction conditions. The reaction mixture is monitored and analyzed to determine the completion of the reaction. The reaction mixture is washed to remove impurities. To the codrug in solution is added a suitable acid. The acid may be added as a solution in the solvent used to dissolve the drug portions, or any other suitable solvent, optionally at a concentration range between 0.1 M and 5 M.

The mixture may be stirred, e.g., for between 3 and 5 hours at room temperature, as the codrug salt crystallizes from the solution, and the resultant crystals may be filter-separated. The crystals may be washed with MTBE or diethyl ether, dried in vacuo, weighed, and assayed for purity. The above process is amenable to large-scale (1 kilogram and greater) process of a codrug salt.

B. Formulations

Another aspect of the present invention provides pharmaceutically acceptable compositions comprising a therapeutically effective amount of a codrug salt as described above, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments, the subject compounds may be simply dissolved or suspended in sterile water. In certain embodiments, the pharmaceutical preparation is non-pyrogenic, i.e., does not elevate the body temperature of a patient.

The phrase “therapeutically effective amount” as used herein means that amount of a compound, material, or composition comprising a codrug salt which is effective for producing some desired therapeutic effect.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The pharmaceutically acceptable salts of the subject compounds include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound that produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

It is known that sterols, such as cholesterol, will form complexes with cyclodextrins. Thus, in preferred embodiments, where the inhibitor is a steroidal alkaloid, it may be formulated with cyclodextrins, such as α-, β- and γ-cyclodextrin, dimethyl-β-cyclodextrin and 2-hydroxypropyl-β-cyclodextrin.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active hedgehog antagonist.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the codrug salts in the proper medium. Absorption enhancers can also be used to increase the flux of the codrug salts across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

In various embodiments, codrug salts can also be administered as a suspension or suspended particles in a gel, such as a hydrogel that is injected, inserted, or implanted; dissolved in polymer matrix and injected, inserted, or implanted; injected into/around bladder, prostrate, bone metastasis, brain, or other tumor site or excised tumor site; incorporated into prosthetic device (e.g., plastic knee or hip) or stent; coated onto prosthetic devices, bone screws, metal plates, etc.; intraaurally administered; applied for any localized painful condition or condition that produces pain; or impregnated into gauzes, wrappings, bandages or dressings.

### EXEMPLIFICATION

The invention now being generally described, it will be more readily understood by reference to the following examples, which are included merely for purposes of illustration of certain aspects and embodiments of the present invention, and are not intended to limit the invention.

### Example 1

### Morphine-diclofenac Codrug Maleate Salt

A morphine-diclofenac codrug (0.865 g) was dissolved in ethyl ether (20 mL). A solution of maleic acid (0.196 g) in ethyl ether (10 mL) was slowly added at room temperature with stirring. The salt which precipitated was immediately filtered off and washed carefully three times with ether (3×15 mL). The solid white product was suspended in 20 mL of deionized water and lyophilized to afford 0.853 g of the morphine-diclofenac maleate as a fine white powder. Alternatively, the filtered salt can dried at 60° C. under high vacuum overnight.

### Example 2

### Morphine-diclofenac Codrug p-toluenesulfonate Salt

A salt was prepared as described above using 0.738 g of the codrug and 0.249 g of p-toluenesulfonic acid in 20 mL of ethyl ether. After lyophilization 0.87 g of the pure salt was obtained.

### Example 3

### Morphine-indomethacin Codrug Maleate Salt

A morphine-indomethacin codrug (0.742 g) was dissolved in 70 mL of ethyl ether and treated dropwise with a solution of maleic acid (0.138 g) in 8 mL of ether. The separated salt was suspended in 20 mL of water and lyophilized to afford 0.806 g of the product.

Those skilled in the art will recognize, or be able to ascertain using no more than routine experimentation, many equivalents to the specific embodiments of the invention described herein. Such equivalents are intended to be encompassed by the following claims.

All references, publications, and patents cited in the specification above are herein incorporated by reference.

1. A pharmaceutically acceptable salt of a codrug, wherein the codrug comprises:
a) a first drug moiety having a first biological activity, or a prodrug form thereof, including a basic nitrogen,
b) a second drug moiety having a second biological activity, or a prodrug form thereof, and
c) a linkage covalently linking said first and second drug moieties to form said codrug, said linkage being cleaved under physiological conditions to regenerate said first and second drug moieties as active agents having said first and second biological activities,
wherein the salt of the codrug has a decomposition rate at room temperature at least 50% lower than the decomposition rate of said codrug as a free base.

2. The salt of a codrug according to claim 1, wherein the first drug moiety is an opioid.

3. The salt of a codrug according to claim 1 or 2, wherein the second drug moiety is an antidepressant compound, an analgesic compound, a steroid, a non-steroidal antiinflammatory drug (NSAIDs), an antibiotic compound, an anti-fungal compound, an antiviral compound, an antiproliferative compound, an antiglaucoma compound, an immunomodulatory compound, a cell transport/mobility impeding agent, a cytokine, a peptide, a protein, an antimetabolite compound, an antipsoriatic compound, a keratolytic compound, an anxiolytic compound, an antipsychotic compound, an alpha-blocker compound, an anti-androgen compound, an anti-cholinergic compound, an adrenergic compound, a purinergic compound, a dopaminergic compound, a vanilloid compound, or an anti-cancer compound.

4. The salt of a codrug according to claim 2, wherein the opioid is morphine or a morphine derivative.

5. The salt of a codrug according to claim 4, wherein the active form of the opioid is represented in the general formula (I): 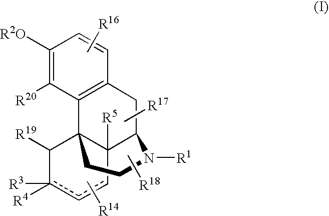
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆alkanoyl group, a C₃₋₆-cycloalkenyl-C₁₋₆-alkanoyl group, an Ar—C₁₋₆-alkyl group, or an allyl group;
R² represents H, a C₁₋₆-alkyl group, or a C₁₋₆-alkanoyl group;
R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R¹⁴ represents a hydrogen, a lower alkyl group, a halogen group, or —C(—OH)(—R¹⁵)₂;
R¹⁵ independently for each occurrence represents a lower alkyl;
R¹⁶, R¹⁷, and R¹⁸ each independently represent a hydrogen, a lower alkyl group, or a halogen group;
R¹⁹ and R²⁰ each represent hydrogen or together represent the oxygen of a dihydrofuran ring;
Ar is phenyl or phenyl substituted by halogen, alkyl C₁₋₃, hydroxyl or alkoxy C₁₋₃; and the dotted line indicates an optional bond.

6. The salt of a codrug according to claim 4, wherein the active form of the opioid is represented in the general formula (II): 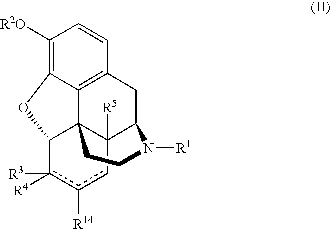
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R² represents a hydrogen, a C₁₋₆-alkyl group, or a C₁₋₆-alkanoyl group;
R³ represents a hydrogen, a C₁₋₆-alkylthio group, an aryl group, a C₄₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R¹⁴ represents a hydrogen, a lower alkyl group, a halogen group, or —C(—OH)(—R¹⁵)₂;
R¹⁵ independently for each occurrence represents a lower alkyl;
Ar is phenyl or phenyl substituted by halogen, alkyl C₁₋₃, hydroxyl or alkoxy C₁₋₃; and the dotted line indicates an optional bond.

7. The salt of a codrug according to claim 4, wherein the active form of the opioid is selected from apomorphine, buprenorphine, codeine, dihydrocodeine, dihydroetorphine, diprenorphine, etorphine, hydrocodone, hydromorphone, levorphanol, meperidine, metopon, o-methylnaltrexone, morphine, naloxone, naltrexone, normorphine, oxycodone, and oxymorphone.

8. The salt of a codrug according to claim 2, wherein the opioid is fentanyl or a fentanyl derivative.

9. The salt of a codrug according to claim 2, wherein the opioid is selected from alfentanil, β-hydroxy-3-methylfentanyl, 4-methoxymethylfentanyl, 4-methyl fentanyl, carfentanil, fentanyl, lofentanil, meperidine, remifentanil, and sufentanil.

10. The salt of a codrug according to claim 2, wherein the active form of the opioid is an analgesic opioid.

11. The salt of a codrug according to claim 3, wherein the second drug moiety is an NSAID.

12. The salt of a codrug according to claim 11, wherein the NSAID is selected from piroxicam, diclofenac, etodolac, indomethacin, ketoralac, oxaprozin, tolmetin, naproxen, flubiprofen, fenoprofen, ketoprofen, ibuprofen, mefenamic acid, sulindac, apazone, phenylbutazone, aspirin, celecoxib and rofecoxib, and derivatives thereof.

13. The salt of a codrug according to claim 11, wherein the active form of the NSAID is diclofenac or a diclofenac derivative.

14. The salt of a codrug according to claim 11, wherein the active form of the NSAID is represented in the general formula (III): 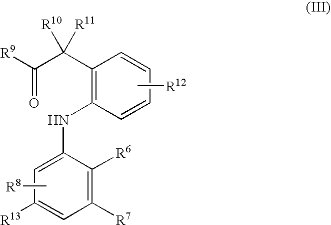
wherein
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R⁹ is OH;
R¹⁰ is a hydrogen or a lower alkyl;
R¹¹ is a hydrogen, a lower alkyl, or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl.

15. The salt of a codrug according to claim 1, wherein the codrug is represented by the general formula (IV): 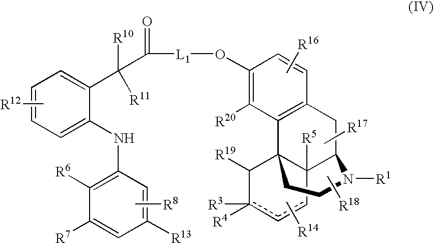
wherein
L₁ is absent or represents a linkage;
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
R¹⁵ independently for each occurrence represents a lower alkyl;
R¹⁶, R¹⁷, and R¹⁸ each independently represent a hydrogen, a lower alkyl group, or a halogen group;
R¹⁹ and R²⁰ each represent hydrogen or together represent the oxygen of a dihydrofuran ring;
Ar is phenyl or phenyl substituted by halogen, alkyl C₁₋₃, hydroxyl or alkoxy C₁₋₃;
and the dotted line indicates an optional bond.

16. The salt of a codrug according to claim 1, wherein the codrug is represented by the general formula (V): 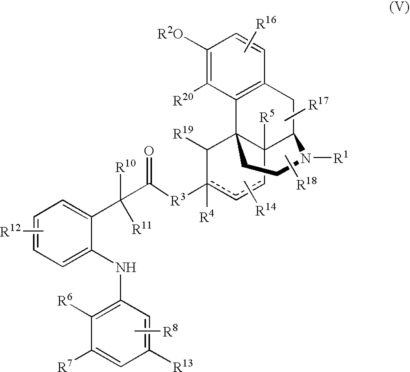
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R² represents a hydrogen, a C₁₋₆-alkyl group, or a C₁₋₆-alkanoyl group;
R³ is absent or represents a linkage;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
R¹⁵ independently for each occurrence represents a lower alkyl;
R¹⁶, R¹⁷, and R¹⁸ each independently represent a hydrogen, a lower alkyl group, or a halogen group;
R¹⁹ and R²⁰ each represent hydrogen or together represent the oxygen of a dihydrofuran ring;
Ar is phenyl or phenyl substituted by halogen, alkyl C₁₋₃, hydroxyl or alkoxy C₁₋₃; and the dotted line indicates an optional bond.

17. The salt of a codrug according to claim 1, wherein the codrug is represented by the general formula (V): 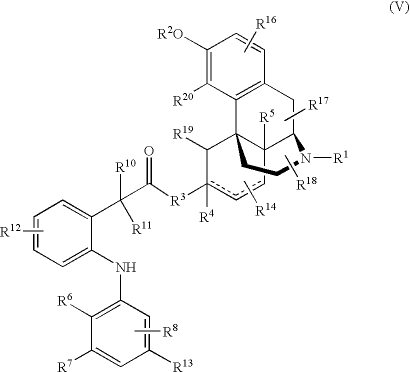
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R² represents a hydrogen, a C₁₋₆-alkyl group, or a C₁₋₆-alkanoyl group;
R³ represents an oxygen, a C₁₋₆-alkylthio group, an aryl group, a C₁₋₆-alkoxycarbonylalkyl group, a C₁₋₆alkyl group, an azido group, a C₁₋₁₂-alkanoyl group, an amine NR^{d}₂ (wherein R^{d}, independently for each occurrence, is hydrogen, a C₁₋₆-alkyl group, or Ar), —C(═O)NH— when R⁴ is a hydrogen, or ═NO— when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
R¹⁵ independently for each occurrence represents a lower alkyl;
R¹⁶, R¹⁷, and R¹⁸ each independently represent a hydrogen, a lower alkyl group, or a halogen group;
R¹⁹ and R²⁰ each represent hydrogen or together represent the oxygen of a dihydrofuran ring;
Ar is phenyl or phenyl substituted by halogen, alkyl C₁₋₃, hydroxyl or alkoxy C₁₋₃; and the dotted line indicates an optional bond.

18. The salt of a codrug according to claim 1, wherein the codrug is represented by the general formula (VI): 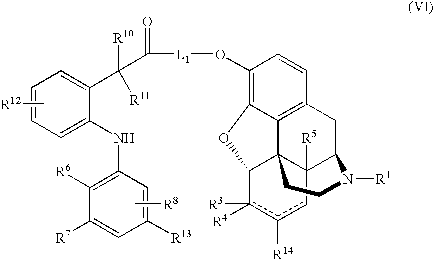
wherein
L₁ is absent or represents a linkage;
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R³ represents a hydrogen, a C₁₋₆-alkylthio group, an aryl group, a C₁₋₆-alkoxycarbonylalkyl group, a C₁₋₆-alkyl group, a hydroxyl group, an azido group, a C₁₋₁₂-alkanoyl group, an amine NR^{d}₂ wherein R^{d} is hydrogen or Ar, or C(═O)NH₂ when R⁴ is a hydrogen, or an oxo group or ═NOH when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
Ar is phenyl or phenyl substituted by halogen, alkyl C₁₋₃, hydroxyl or alkoxy C₁₋₃;
and the dotted line indicates an optional bond.

19. The salt of a codrug according to claim 1, wherein the codrug is represented by the general formula (VII): 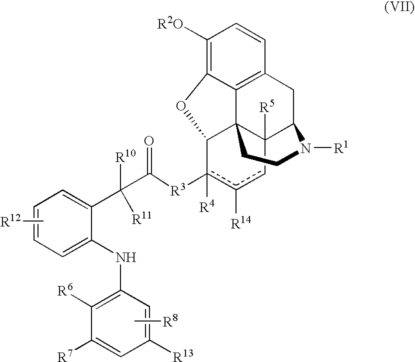
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₆-cycloalkenyl-C₁₋₆-alkanoyl group, an Ar—C₁₋₆-alkyl group, or an allyl group;
R² represents a hydrogen, a C₁₋₆-alkyl group, or a C₁₋₆-alkanoyl group;
R³ is absent or represents a linkage;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R₅ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
Ar is phenyl or phenyl substituted by halogen, alkyl C₁₋₃, hydroxyl or alkoxy C₁₋₃;
and the dotted line indicates an optional bond.

20. The salt of a codrug according to claim 1, wherein the codrug is represented by the general formula (VII): 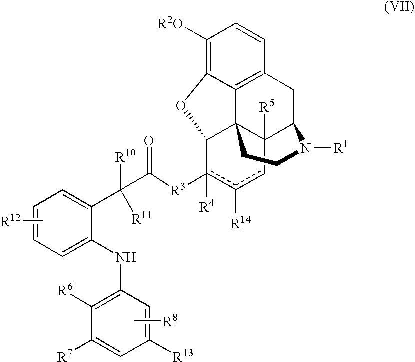
wherein
R¹ represents a hydrogen, a C₁₋₆-alkyl group, a C₃₋₆-cycloalkyl-C₁₋₆-alkyl group, a C₁₋₆-alkenyl group, a C₁₋₆-alkanoyl group, a C₃₋₆-cycloalkenyl-C₁₋₆-alkyl group, a C₃₋₆-cycloalkyl-C₁₋₆-alkanoyl group, a C₃₋₄-cycloalkenyl-C₁₋₆-alkanoyl group, an Ar—C₁₋₆-alkyl group, or an allyl group;
R² represents a hydrogen, a C₁₋₆-alkyl group, or a C₁₋₆-alkanoyl group;
R³ represents a C₁₋₆-alkylthio group, an aryl group, a C₁₋₆-alkoxycarbonylalkyl group, a C₁₋₆-alkyl group, an oxygen, an azido group, a C₁₋₁₂-alkanoyl group, an amine NR^{d}₂ (wherein R^{d}, independently for each occurrence, is hydrogen, a C₁₋₆-alkyl group, or Ar), —C(═O)NH— when R⁴ is a hydrogen, or ═NO— when R⁴ is absent;
R⁴ is absent or represents a hydrogen;
R⁵ represents a hydrogen, a hydroxyl group, a lower alkyl group, an amine NRₐR_{b} wherein Rₐ is a hydrogen, alkyl C₁₋₁₂, alkenyl C₃₋₈, cycloalkyl C₃₋₇ alkyl C₁₋₄, Ar-alkyl C₁₋₅ or Ar-alkenyl C₃₋₅, provided that Rₐ does not contain the system —CH═CH— attached to the nitrogen atom; and R_{b} is hydrogen, alkyl C₁₋₈, or the group COR_{c} wherein R_{c} is a hydrogen, alkyl C₁₋₁₁, alkenyl C₂₋₇, Ar, Ar-alkyl C₁₋₅, Ar-alkenyl C₂₋₅, cycloalkyl C₃₋₈, or cycloalkyl C₃₋₈ alkyl C₁₋₃;
or R⁴ and R⁵ taken together represent —(CH₂)₂—;
R⁶ is a lower alkyl, a lower alkoxy, a fluoro, or a chloro;
R⁷ and R⁸ are each, independently for each occurrence, a hydrogen, a lower alkyl, a fluoro, a chloro, or a trifluoromethyl;
R¹⁰ is a hydrogen or a lower alkyl;
R¹¹ is a hydrogen, a lower alkyl or when R¹⁰ is hydrogen, benzyl;
R¹² is a hydrogen, a lower alkyl, a lower alkoxy, a fluoro, a chloro, or a bromo;
R¹³ is hydrogen or trifluoromethyl when R⁶ is hydrogen or chloro and R⁷ is hydrogen or trifluoromethyl;
R¹⁴ represents a hydrogen, a lower alkyl group, a halogen group, or —C(—OH)(—R¹⁵)₂;
Ar is phenyl or phenyl substituted by halogen, alkyl C₁₋₃, hydroxyl or alkoxy C₁₋₃; and the dotted line indicates an optional bond.

21. The salt of a codrug according to claim 17 or 20, wherein R³ represents a C₁₋₆-alkylthio group, an aryl group, a C₁₋₆-alkoxycarbonylalkyl group, a C₁₋₆-alkyl group, an oxygen, an azido group, a C₁₋₁₂-alkanoyl group, an amine NR^{d}₂ (wherein R^{d}, independently for each occurrence, is hydrogen or Ar), —C(═O)NH— when R⁴ is a hydrogen, or ═NO— when R⁴ is absent.

22. The salt of a codrug according to claim 2, wherein the first drug moiety is morphine and the second drug moiety is diclofenac.

23. The salt of a codrug according to claim 1, wherein the linkage is hydrolyzed in bodily fluid.

24. The salt of a codrug according to claim 20, wherein the linkage includes one or more hydrolyzable groups selected from an ester, an amide, a carbamate, a carbonate, a cyclic ketal, a thioester, a thioamide, a thiocarbamate, a thiocarbonate, a xanthate and a phosphate ester.

25. The salt of a codrug according to claim 1, wherein the linkage is enzymatically cleaved.

26. The salt of a codrug according to claim 1, wherein the linkage includes a polyethylene glycol, a glycerol, a sugar, an alkylene chain, an amino acid, or an oligopeptide.

27. The salt of a codrug according to claim 1, wherein said codrug salt includes a counterion capable of protonating the basic amine.

28. The salt of a codrug according to claim 1, wherein said codrug salt is formulated from an organic acid.

29. The salt of a codrug according to claim 25, wherein said organic acid is selected from maleic acid, malonic acid, oxalic acid, tartaric acid, citric acid, lactic acid, fumaric acid, benzoic acid, p-toluenesulfonic acid, methanesulfonic acid, acetic acid, adipic acid, formic acid, and salicylic acid.

30. The salt of a codrug according to claim 1, wherein said codrug salt is formulated from an inorganic acid.

31. The salt of a codrug according to claim 27, wherein said inorganic acid is selected from hydrochloric acid, sulfuric acid, hydrobromic acid, nitric acid, and phosphoric acid.

32. The salt of a codrug according to claim 1, wherein the salt of the codrug has a decomposition rate at room temperature less than 10% of the decomposition rate of said codrug as a free base.

33. The salt of a codrug according to claim 1, wherein the salt of the codrug has a decomposition rate at room temperature less than 1% of the decomposition rate of said codrug as a free base.

34. The salt of a codrug according to claim 1, wherein the codrug has an ED₅₀ for each of said first and second biological activities at least 10 times greater than the ED₅₀ of said regenerated first and second drug moieties as active agents.

35. The salt of a codrug according to claim 1, wherein the codrug has an ED₅₀ for each of said first and second biological activities at least 1000 times greater than the ED₅₀ of said regenerated first and second drug moieties as active agents.

36. The salt of a codrug according to claim 1, which is essentially insoluble in body fluids.

37. The salt of a codrug according to claim 32, wherein said regenerated first and second drug moieties are at least 10 times more soluble than said codrug salt.

38. A malonic acid salt of a codrug comprising morphine covalently linked to diclofenac by a bond which is hydrolyzable to regenerate active morphine and diclofenac in vivo.

39. The salt of a codrug according to claim 1, wherein the salt includes the codrug and a pharmaceutically active counterion.

40. A method of manufacturing a salt of a codrug according to claim 1, comprising conjugating an opioid and an NSAID, and crystallizing the codrug salt.

41. A pharmaceutical composition of a codrug salt according to claim 1, wherein the codrug salt is combined with a pharmaceutically acceptable excipient.

42. The salt of a codrug according to claim 1, wherein the codrug salt is dispersed in a hydrogel.

43. The salt of a codrug according to claim 1, wherein the codrug salt has a purity greater than the purity of the codrug as a free base.

44. The salt of a codrug according to claim 40, wherein the codrug salt has a purity of at least 97%.

45. A pharmaceutically acceptable salt of a codrug according to claim 1, wherein at least three drugs are linked to one another covalently.

46. A pharmaceutically acceptable salt of a codrug according to claim 1, wherein release of the active drugs follows pseudo-zero-order kinetics.

47. A pharmaceutically acceptable salt of a codrug according to claim 1, wherein the drugs are covalently linked and release of the active drugs follows pseudo-zero-order kinetics for about 10 days to about 6 weeks.

48. A pharmaceutically acceptable salt of a codrug according to claim 44, wherein release of each active drug follows pseudo-zero-order kinetics for about 3 weeks.

49. A pharmaceutically acceptable salt of a codrug according to claim 1, which is soluble in body fluids.

50. A pharmaceutically acceptable salt of a codrug according to claim 1, wherein said salt form is formulated from an acid that treats at least one symptom of a condition.

51. A method of relieving pain comprising administering an effective amount of a pharmaceutically acceptable salt of a codrug according to claim 1 to a patient in need of pain relief.

52. The salt of a codrug according to claim 1, wherein the first and second drug moieties are different.

53. The salt of a codrug according to claim 1, wherein the first and second drug moieties are the same.
